(19) United States
(12) Patent Application Publication
(10) Pub. No.: US 2018/0116525 A1
(43) Pub. Date: May 3, 2018

(54) FLEXIBLE, NON-INVASIVE REAL-TIME HEMATOMA MONITORING SYSTEM USING NEAR-INFRARED SPECTROSCOPY

(71) Applicant: The Regents of the University of California, Oakland, CA (US)

(72) Inventors: Claire Meyer Lochner, Moraga, CA (US); Rachel Nancollas, Layfayette, CA (US); Jacob Sadie, Berkeley, CA (US); Yasser Khan, Oakland, CA (US); Ana Claudia Arias, Oakland, CA (US)

(21) Appl. No.: 15/852,366

(22) Filed: Dec. 22, 2017

Related U.S. Application Data
(63) Continuation of application No. PCT/US2016/039406, filed on Jun. 24, 2016.
(60) Provisional application No. 62/183,843, filed on Jun. 24, 2015.

(51) Int. Cl. A61B 5/00

(52) U.S. Cl. CPC A61B 5/0075; A61B 5/0024; A61B 5/0064; A61B 5/0086; A61B 5/4869; A61B 5/0042

(57) ABSTRACT

NIRS systems including fully-printed LEDs and PDs operating in the NIR spectrum are provided that provide conformal adherence to a patient's head and efficient measurement of near-infrared absorption. Circuitry processes and wirelessly transmits data from one or multiple devices located on a patients head to a remote and/or portable device. Using multiple sensors improves the spatial resolution of the measurements, and post processing on the portable device provides the ability to transmit results to a patient's physician, e.g., via a secure internet connection.

This invention was made with government support under Grant Number DGE1106400 awarded by the National Science Foundation. The government has certain rights in the invention.

### BACKGROUND

The presence of hematomas (e.g., epidural, subdural, and intracranial) as a result of blunt head trauma or stroke can lead to lasting brain damage or even death if not treated in a timely manner. Monitoring at-risk patients is critical for the detection and treatment of hematomas. CT and MRI scans are commonly used to assess the severity and location of hematomas, but many patients, especially those in developing nations or on the battlefield, do not have access to these technologies. Near-infrared spectroscopy (NIRS) has been demonstrated as a facile technique for hematoma detection in all levels of the brain and is a cheap, non-invasive alternative to CT and MM scans. However, many limitations exist that prevent NIRS from being the gold-standard diagnostic technique. Those limitations include optical contact issues on the surface of the head, penetration depth capabilities of selected light sources, and the ability to perform real-time monitoring of a patient's condition.

Also, current portable or handheld technologies for hematoma detection have poor spatial resolution, poor penetration depth, and are generally used for one-time diagnosis of hematoma presence. Both single scans and continued monitoring of hematomas are applications of interest, and there is plenty of room for improvement in each area. Emergency rooms, intensive care units, military field hospitals, ambulances, etc. are all locations where hematoma monitoring may be critical to patient care. In addition, postoperative chronic hematoma patients would greatly benefit from predictive monitoring outside of a clinical setting.

Currently there are no applications using NIRS in sensor networks. Despite the lack of development in this area, sensor networks could improve the ability to predict hematomas, particularly in the case of bilateral hematomas that are often missed due to the use of a patient's body as the control. Wireless sensor networks have been developed for other types of body sensors such as EEGs. Currently, there is only one commercial on-chip sensor network interface called Sensium. However, since its primary application is EEG measurements, it works at much higher frequencies and resolution (and thus uses more power) than is required for NIRS. There are several approaches that use multi-chip solutions, however these tend to be power inefficient because of the difficulty in tailoring commercial off-the-shelf (COTS) components to provide a limited, specific set of functions. Therefore, the power performance of existing sensor networks could be drastically improved if such networks were designed for NIR sensors.

### SUMMARY

The present disclosure provides novel NIRS systems using cheap, flexible, large-area light emitting diodes (LEDs) and photodiodes (PDs) and which provide conformal adherence to a patient's head and efficient measurement of near-infrared absorption. Using fully-printed LEDs and PDs operating in the NIR spectrum according to certain embodiments, such NIRS systems have potential applications beyond hematoma sensing, such as pulse oximetry or night-vision. In certain embodiments, circuitry is provided to process and wirelessly transmit data from one or multiple devices located on a patients head to a remote and/or portable device. Using multiple sensors improves the spatial resolution of the measurements, and post processing on the portable device provides the ability to transmit results to a patient's physician, e.g., via a secure internet connection. The embodiments herein provide an improvement over currently available handheld hematoma sensors for some or all of the following reasons: (1) the ability of the flexible light sources and sensors to more conformally fit to the patient's head, (2) multiple concurrent sensing locations provides better data reliability and signal-to-noise ratio (SNR), and (3) continuous monitoring provided by mounted sensors increases the chances of detecting a hematoma in a timely manner.

According to an embodiment, a wearable sensor device is provided that typically includes a flexible substrate, and an array of organic near infra-red (NIR) elements formed on the flexible substrate, wherein the array of NIR elements includes at least one organic NIR light emitting element and at least one organic NIR light detection element. In certain aspects, the wearable sensor device is a hematoma detection device. In certain aspects, the array of NIR elements includes at least one organic light emitting diode (LED) element and at least one organic photodetector (OPD) element. In certain aspects, the NIR elements include an array of NIR LEDs and an array of NIR OPDs. In certain aspects, the flexible substrate comprises a plastic material. In certain aspects, the plastic material includes one of a polyimide (PI) film, a polyethylene terephthalate (PET) film or a polyethylene naphthalate (PEN) film. In certain aspects, the flexible substrate comprises a parylene layer formed on a flexible backing layer. In certain aspects, the device further includes one or more printed batteries formed on the flexible substrate or on the array of organic NIR elements. In certain aspects, the device further includes a transmitter and control element for wirelessly communicating with a remote device. In certain aspects, the transmitter and control element includes a low noise amplifier, an analog to digital converter, and a wireless transmission circuit. In certain aspects, the wireless transmission circuit includes or is coupled to a printed antenna element formed on the flexible substrate or on the array of organic NIR elements.

According to another embodiment, a sensor network system is provided that typically includes a processing unit and at least two wearable sensor devices, each wearable sensor device including an array of organic near infra-red (NIR) elements formed on a flexible substrate, wherein the array of NIR elements includes at least one organic NIR light emitting element and at least one organic NIR light detection element. The network system also typically includes a wireless communication element connected to the at least one organic NIR light detection element, wherein the communication element receives a signal from the at least one NIR light detection element and sends a corresponding sensor signal to the processing unit. The processing unit receives the sensor signals from the at least two wearable sensor devices and stores the sensor signals and/or data derived from the sensor signals, to a memory. In certain aspects, the sensor network system is a hematoma sensor network system. In certain aspects, each array of NIR elements includes at least one organic light emitting diode (LED) element and at least one organic photodetector (OPD) element. In certain aspects, each flexible substrate comprises a plastic material selected from the group consisting of a polyimide (PI) film, a polyethylene terephthalate (PET) film and a polyethylene naphthalate (PEN) film.

According to a further embodiment, a method is provided for forming a detection device. The method typically includes providing a flexible substrate, and forming an array of organic near infra-red (NIR) elements on the flexible substrate, wherein the array of NIR elements includes at least one organic NIR light emitting element and at least one organic NIR light detection element. In certain aspects, the detection device is a wearable sensor device. In certain aspects, the detection device is a hematoma detection device. In certain aspects, forming an array includes printing said NIR elements on the flexible substrate. In certain aspects, printing includes one or more of ink jet printing, screen printing and blade coating. In certain aspects, the flexible substrate comprises a plastic material selected from the group consisting of a polyimide (PI) film, a polyethylene terephthalate (PET) film and a polyethylene naphthalate (PEN) film. In certain aspects, forming an array includes using photolithography techniques to form said NIR elements on the flexible substrate.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

The detailed description is described with reference to the accompanying figures. The use of the same reference numbers in different instances in the description and the figures may indicate similar or identical items.

FIG. 1 illustrates the absorption coefficients for water, oxyhemoglobin, and deoxyhemoglobin as a function of wavelength.

FIG. 2 shows a side view schematic of LED/PD structure according to an embodiment.

FIG. 3 illustrates the comparison of solution processing techniques to be used to device layer fabrication.

FIG. 4 shows a facile device patterning materials.

FIG. 5 illustrates a diagram summarizing material properties for PEN and PET.

FIG. 6 shows a model network of hematoma sensors wirelessly connected to a remote, belt-mounted processing unit according to en embodiment.

FIG. 7 shows a system diagram of LED, photodiode, and controlling circuitry according to an embodiment.

FIG. 8 illustrates a waist-mounted sensor processing unit consisting of board printed antenna, COTS RF front end, microcontroller, memory, and 802.11 transceiver for transmitting data to medical personnel according to an embodiment.

FIG. 9 shows examples of fully assembled sensors composed of NIR OLEDs and/or OPDs according to an embodiment: FIG. 9A shows a printed NIR organic LED (OLED) array on flexible PEN substrate, FIG. 9B shows a printed organic photodetector (OPD) array on flexible PEN substrate, and FIG. 9C shows a flexible sensor composed of assembled OLED and OPD arrays.

FIG. 10 shows an OPD signal upon excitation by light from NIR OLEDs that is reflected from a subject's arm according to an embodiment.

FIG. 11 shows how the reflected intensity of light at the sensor can vary over the area covered by each of its pixels.

### DETAILED DESCRIPTION

Printed electronics technologies have enabled circuits to rapidly transition from rigid substrates to flexible, conformal substrates, opening a wide array of previously unavailable applications including body-deployed sensors. Furthermore, the cost associated with printed circuits is dramatically lower than traditional ICs with regard to materials, substrates, and equipment expenses. Taking advantage of these maturing processes, in certain embodiments, printed LEDs and PDs are fabricated on conformal substrate(s) to provide devices that enable rapid detection of the presence of hematomas in the human brain using the near-infrared spectrum. NIRS is an effective technique for detecting hematomas as deep as a few centimeters inside the brain. The alternatives to NIRS are MRI and CT imaging, which are considerably more expensive and unavailable in a number of environments where hematoma detection is critical. NIRS techniques typically use two large sensors, one on each side of the brain, and the patient behaves as his own control. While this is effective, improving the sensor count around the skull will greatly improve the spatial resolution of the hematoma mapping. Finally, the sensor devices, in certain embodiments, are flexible, built on bandage-like substrates, and can also be linked to wireless networks for remote and real-time monitoring. The array of sensors (e.g., ASIC sensors) on a single patient will feed readings into a single, processing unit or system capable of signal processing and wireless communication. The present disclosure provides device embodiments that are insensitive to poor optical contact, highly networked and spatially resolute, and wirelessly connected for real-time monitoring capabilities.

Printed LEDs and PDs on flexible substrates advantageously overcome optical contact limitations. Printed devices can provide sufficient performance at a much lower manufacturing cost. Furthermore, the transition to a flexible substrate and flexible technology provides a much higher penetration depth and an improved signal-to-noise ratio (SNR) when compared to current technologies. Additionally, the spatial resolution of hematoma sensing is improved through multiple networked patches. Large-area printed LEDs and PDs enable the facile production of multiple-sensor bandages, capable of being linked together in an arrayed manner to more accurately locate and assess hematoma presence within the brain. In one embodiment, a wirelessly-linked patch sensor network for real-time monitoring applications is provided.

Early detection of hematomas in injured patients may be critical for the prevention of long-term brain injuries that would drastically degrade a patient's quality of life or potentially lead to death.

### Physics

The underlying principle used for hematoma detection is the difference in optical density of two normally identical locations in the brain. Taking advantage of the expected physiological symmetry in the brain, hematoma detection protocols generally use a single patient as both the experiment and control sample by comparing the optical density of reflected light from hemoglobin present in blood on either side of the brain. The optical density is simply a measure of the ratio of light intensity transmitted into versus light intensity reflected from the brain, as described in equation 1. If a significant difference in optical density is present, it is highly likely that a hematoma is present in the side of the brain with the higher optical density reading.

$$OD = log ( \frac{{I}_{o}}{I} ) \qquad ( 1 )$$

An optical density measurement is performed by shining light at an appropriate wavelength into the tissue (e.g., brain) and measuring the intensity of the light reflected at the same wavelength. In hematoma detection applications, the standard approach is to determine the amount of light reflected by hemoglobin present in blood. If the concentration of hemoglobin in blood is constant, an increase in blood volume results in an increase in hemoglobin count. This increases light absorption, which is indicated by a higher optical density measurement on the side of the brain with hematoma.

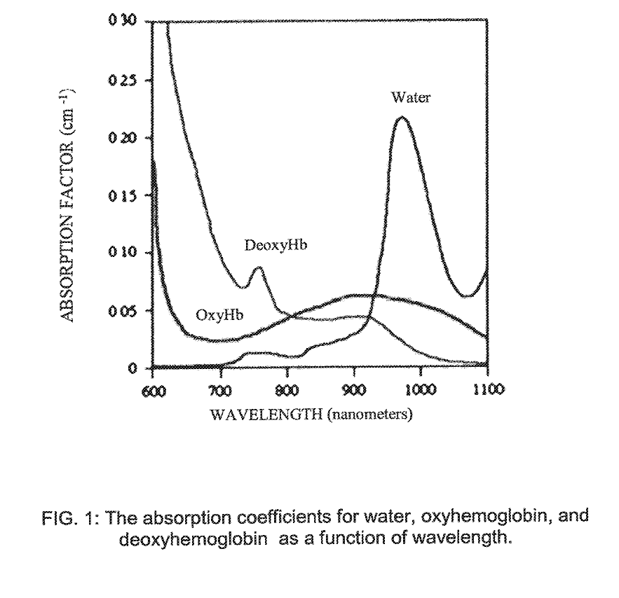

The three main materials in the body one must consider when performing a NIRS measurement are water, oxyhemoglobin, and deoxyhemoglobin. In order to avoid confounding responses, it is critical to select a wavelength for the measurement that provides dramatically different absorption coefficients for each of the materials. As shown in FIG. 1, a wavelength within the range of 700-1000 nm is a suitable option. Specifically, a wavelength of approximately 800 nm would provide an equivalent response for both oxyhemoglobin and deoxyhemoglobin while the response from water would be nearly nonexistent.

### Printing

Two major issues identified with the current technologies available are a) those of optical contact being compromised by non-perpendicular measurements and b) probe motion during measurement degrading SNR. According to various embodiments, to circumvent these issues a conformal device is positioned on a patient's head, both fixing the probe location and ensuring a measurement perpendicular to the surface of the skin. Printed deposition techniques are used in certain embodiments to fabricate such devices. Gravure, inkjet, and screen printing along with blade coating offer flexibility in the design process and material selection for diodes as well as detectors. All of these components are easily fabricated on a variety of substrates that can flex, bend, stretch, and shift to accommodate the various contours of the human head.

### Multiplexing and Monitoring

As explained above, optical density difference is a key indicator of hematoma presence, which implies at least two measurements are required to provide the appropriate diagnosis. While prior devices allow for measurements in multiple locations, they lack the ability to monitor each of these locations over a particular amount of time without performing multiple diagnoses. In certain embodiments, a device is provided that is adhered to the skull for temporary monitoring that alleviates the need for multiple time-consuming measurements on a patient. Furthermore, wirelessly linking of multiple devices provides the ability to continuously monitor the optical density and observe the development of hematomas.

### Sensor Circuitry

There are currently no other NIRS systems that use system-on-a-chip (SoC) solutions. In an embodiment, an application-specific integrated circuit (ASIC) solution is provided in which fewer rigid chips are needed, which allows for improved optical contact. Moreover, a SoC solution is more power efficient because it need not contain unnecessary features. Also, use of a custom wireless communication bypasses commercial wireless protocols, which tend to have high peak current consumption. Power efficiency and low peak currents allow the use of printed batteries for powering sensor networks. Not only are printed batteries conformal, providing better optical contact, but they also remove safety concerns associated with fixing batteries to the skin such as chemical reactions, explosion, and heating. Certain embodiments employ printed batteries.

### Driving Innovation and Improving Public Health

A real-time monitoring device for hematoma presence will help propel both the cloud-based patient care and personal medicine industries. For example, physicians will advantageously be able to monitor a number of patients from a distance, and alarms can be triggered when optical density measurements fall outside nominal values, helping prevent a number of traumatic, life-altering injuries. As the cost of certain device embodiments should be small, the devices could be used for both simple screening tests as well as long-term monitoring for patients with chronic issues and postoperative patients at risk for hematoma. Such devices will also enable a low-cost, body-deployed technique for mapping and understanding how hematomas develop as a function of various traumas to the head, furthering medical knowledge and promoting preventative techniques.

A NIR hematoma sensor has the potential to bridge the gap between the next generation of electronics and medical technology. Hematomas may develop gradually, without symptoms, until permanent damage is already done. The NIRS hematoma sensor embodiments will provide medical staff with the ability to remotely and continually monitor their patients for hematoma development. The various device embodiments will reduce the need for expensive CT scans and provide patients at-risk for hematomas after head injury and/or surgery with a low cost and non-invasive wearable device that will alert them and their caretakers to the presence of a developing hematoma. The faster the diagnosis of a hematoma, the better the medical outcome. Finally, the method embodiments for fabricating printed NIR LEDs and PDs herein are useful to create printed devices at similar wavelengths for other NIRS-based medical monitoring applications such as pulse oximetry and targeted drug delivery.

In addition to developing printed LEDs and PDs, the present disclosure provides a NIRS system that uses a wireless network of sensors, e.g., SoC sensors. Leveraging the extremely low duty cycle required for NIR measurements, using ASICs, and using custom wireless protocols allows for an extremely low power budget and maximum current.

### LED and PD Materials Development

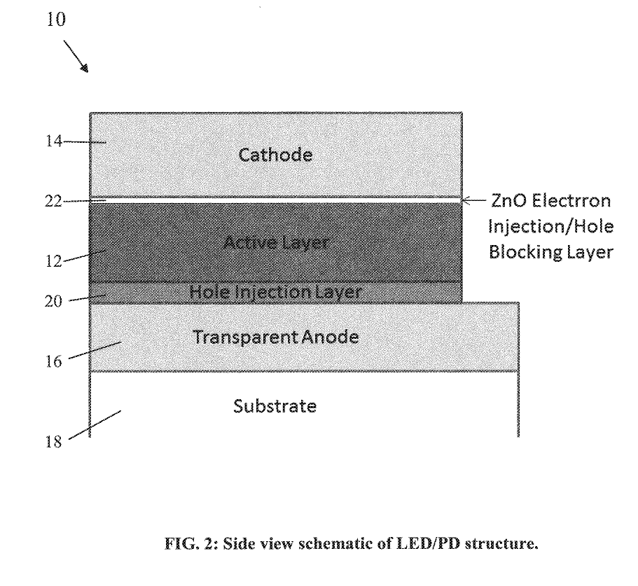

FIG. 2 illustrates a side view of a LED/PD structure 10 according to an embodiment. As shown, structure 10 includes an active layer 12, e.g., a photo-responsive or photo-emissive material layer, and a cathode layer 14 and anode layer 16. Cathode layer 14 and anode layer 16 are shown as being positioned on opposite sides of active layer 12, however, it will be appreciated that anode 16 and cathode 14 may be located on the same side/surface of active layer 12, Optional hole injection layer 20 and hole blocking layer 22 are also shown. Substrate 18 offers structural stability, e.g., during device formation and use. Both organic and inorganic materials have been successfully developed for use in solution-processed photo-sensing applications and may be used in various embodiments. In certain embodiments, inorganic materials are used because while organic materials functioning in the visible wavelength regime are commercially available, organic molecules that emit or absorb in the near infrared such as platinum porphyrins require difficult synthesis with low yields. Inorganic materials with emission and absorbance peaks in the NIR wavelengths have higher synthetic yields and are thus more readily accessible than organic materials with similar emission and absorption characteristics. One such inorganic material includes PbS quantum dots. PbS quantum dots are solution-processable and can function as either tunable emitters or absorbers, with peak emission and absorption wavelengths ranging from the visible to the infrared. The characteristic wavelengths of quantum dots can be accurately predicted through proper quantum dot radius size control, making quantum dots an excellent material for NIR LEDs and PDs for a hematoma sensor application.

Bright infrared quantum dot LEDs (QLEDs) with direct electrical excitation have been demonstrated by Sun et al. (L. Sun, et al. Bright infrared quantum-dot light-emitting diodes through inter-dot spacing control. Nat. Nano., 7: 369373, 2012). These QLEDs operate at low voltages with 6.4

$$\frac{W}{sr \cdot {m}^{2}}$$

radiance, 2% external quantum efficiency, and peak emission ranging from 900-1800 nm. This record-setting IRQLED performance was achieved by engineering ligands on the quantum dots surface to control inter-dot spacing, assisting efficient charge transport and radiative exciton recombination within the device active layer. In order to achieve similar QLED operation at 800 nm, PbS quantum dots with a smaller radius are used in order to blue-shift the emission peak. Furthermore, recent ligand-stripping and exchange techniques [E. L. Rosen, et al. Exceptionally Mild Reactive Stripping of Native Ligands from Nanocrystal Surfaces by Using Meerweins Salt. Angewandte Chemie, 51(3): 684-689, 2012] can be applied to improve the LED efficiency. The adaptation of this ligand exchange approach is expected to boost LED quantum efficiency by more effectively transferring the shorter ligands to the nanoparticle surface and reducing oleate contamination most likely present in the process developed by Sun et al.

In an embodiment, a QLED has a PbS/CdS doped polymer active layer 12. For the QLED, quantum dots with the appropriate radius for 800 nm peak are used in an embodiment. Poly[2-methoxy-5-(2′-ethylhexyloxy-p-phenylenevinylene)] (MEH-PPV), the polymer host, commercially available from Sigma Aldrich, has a lower ionization potential than PbS/CdS quantum dots, making charge separation at the polymer/QD interface and the transfer of holes to the hole-transport-dominated polymer favorable. In another embodiment for an OLED device, emissive layer 12 includes a solution processed phosphorescent NIR emitter material made by Cambridge Display Technology. This material can be spin-coated or blade-coated from an o-xylene solution. For an OPD device, active layer material 12 may be spin-coated or blade-coated from a PCDTBT:PC₇₁BM (where PCDTBT is a polymer and PC₇₁BM is a fullerene) solution or Cambridge Display Technology's NIR-absorbing bulk heterojunction photoactive material.

Useful materials for cathode 14 include lithium fluoride, calcium, and aluminum, which must be evaporated. Higher work function metals such as silver can be printed, but create a larger barrier for electron currents that limits device performance. In certain embodiments, a polyethyleneimine (PEI) surface treatment demonstrated by Zhou et al. (Y. Zhou. A Universal Method to Produce Low Work Function Electrodes for Organic Electronics. Science, 336(6097): 327-332, 2012.) is used to lower the work function of the silver cathode by 1 eV. For the anode 16, indium tin oxide (ITO) is used. While suitable for devices built on glass, ITO tends to crack when bent and so it may not be an ideal anode material for a flexible device. In certain embodiments, either organic materials such as PEDOT:PSS and/or inorganic materials such as transparent conducting oxides (e.g., WO₃ printed from nanoparticle ink) or ultrathin metal layers (e.g., Ag printed from organo-metallic precursor ink) are used. A highly conductive PEDOT:PSS PH1000 ink is commercially available and can be deposited over a silver nanowire mesh. The silver nanowires are very conductive and have superior optical transparency in the near infrared region compared to ITO. Other printed transparent conducting oxides may be used as well as solution-processable materials including intrinsic oxides, doped oxides, and nanomaterials systems. In addition, gold films may be used as they are transparent when deposited at thicknesses below 10 nm.

The QLEDs and PDs may be fabricated using standard methods, e.g., spin coat the active layers on a substrate and thermally evaporate the top contact.

### Printing

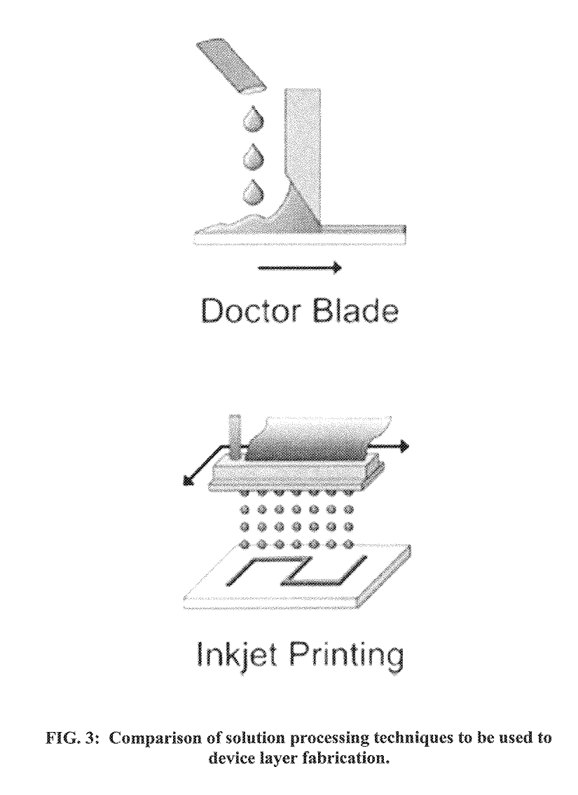

In an embodiment, processes for forming QLEDs and OPDs include printing techniques such as, for example, inkjet printing and blade coating. Smaller area features such as the anode and cathode electrodes 14, 16 may be deposited from nanoparticle inks via inkjet printing as depicted in FIG. 3. In one embodiment, a blade coating method (see FIG. 3) is used to print the larger active areas of the LEDs and PDs. Blade coating is useful to print large surface areas with minimal source material and has already been successfully demonstrated for LED fabrication. Defect-free uniform and repeatable films can be achieved with blade coating by using a computer controlled actuator to move the blade relative to the substrate while an actuated syringe pump dispenses solution in front of the blade.

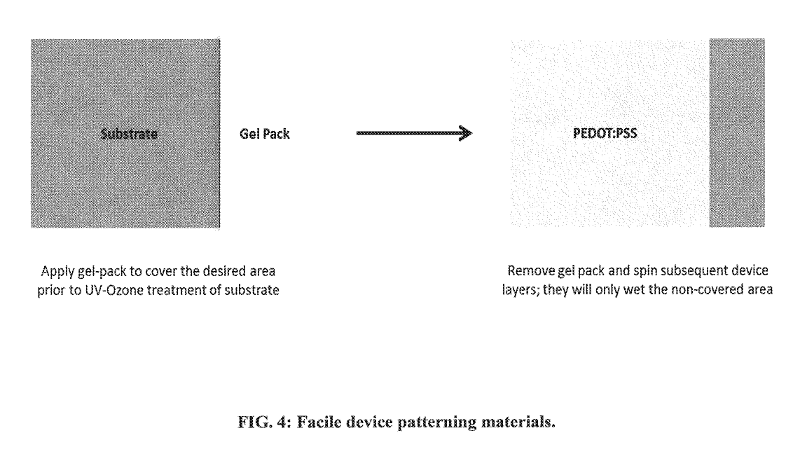

To demonstrate successful multi-layer solution processing, layer-by-layer fabrication with carefully selected orthogonal solvent systems is performed in order to avoid dissolution of previously deposited layers. Test device anode patterning will be performed by applying a gel pack coating material that adheres to the substrate via van der Waals interaction, leaving no contamination on the surface after removal. While masked, UV-ozone treatment will selectively alter the surface energy of our exposed substrate. Next, PEDOT:PSS suspended in water, the first layer of both the LEDs and PDs, will be printed with the blade coater. Due to the surface energy difference imposed by the selective UV-ozone treatment, the PEDOT:PSS will adhere only to the hydrophilic area of the substrate (see FIG. 4 for demonstration of self-aligning PEDOT:PSS). Then, quantum dots and MEH-PPV suspended in o-xylene or phosphorescent NIR material dissolved in o-xylene (for LED fabrication) or a bulk heterojunction polymer:fullerene blend suspended in organic solvents (for PD fabrication) will be deposited onto the PEDOT:PSS, followed by PEI suspended in methoxyethanol. Finally, a cathode layer is deposited using conductive silver inks. Many metallic inks require thermal activation in order to establish high conductivity, however large thermal treatments may irreversibly damage the already-deposited quantum dot layer. Magdassi et al. have demonstrated room temperature silver nanoparticle sintering, mitigating this issue [46]. In this approach, silver nanoparticles spontaneously sinter upon contact with ink jet printed PDAC, a polycation material that alters the surface properties of the silver nanoparticles via charge neutralization and possibly the desorption of the nanoparticle stabilizer. This process results in a very conductive silver film with 0.05 Ω/s sheet resistance, without harming the active layer of the PDs and LEDs. Another low-temperature compatible cathode deposition technique includes printing InkTec's silver organo-metallic precursor ink. This commercially available Ag precursor ink can be deposited via spin-coating, gravure printing, or blade coating. Once deposited, the ink forms a highly smooth, reflective, and conductive film after being annealed at 120° C. for 5 minutes.

### Substrates

In certain embodiments, to overcome optical contact issues, printed devices include flexible substrates, which advantageously have the ability to conform to the surface of interest. Transitioning to such a substrate poses challenges on both a practical and performance level. For example, when dealing with printing processes, the surface energy of the substrate will determine the critical dimensions of various inks. The coffee ring effect is also dependent on surface energy and results in the redistribution of particulate material in an ink toward the perimeter of the deposited feature. Therefore, the surface energy of a substrate will determine how much wetting/spreading occurs for inks and how deposited material will be distributed, both of which will impact device performance and lifetime. Furthermore, substrate selection may limit the thermal budget of subsequent processing steps. With nanoparticle-based metal inks, for example, thermal treatments are necessary for establishing highly conductive paths on the substrate, but these thermal treatments should be designed to successfully sinter films below the glass transition temperature of the substrate.

To mitigate a few of the surface property issues, thin layers of parylene are deposited onto substrates in order to improve the printing performance and feature morphology. In certain embodiments, low sinter temperature nanoparticle inks, for example gold and silver inks, are used for many material systems.

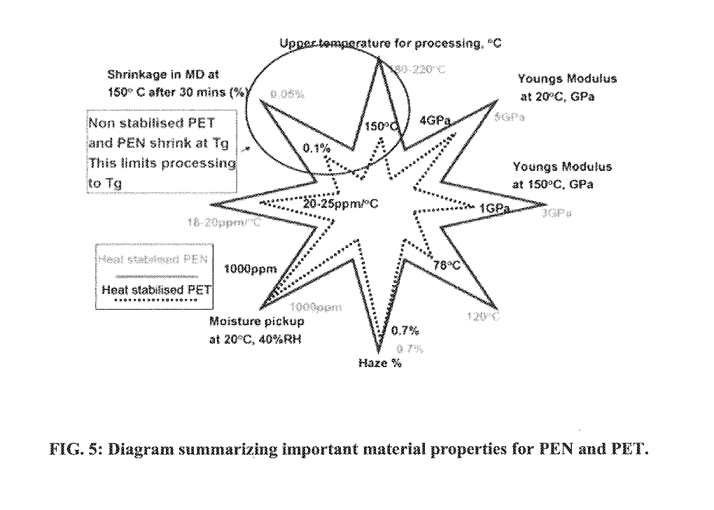

Useful flexible substrate materials for solution-processed electronics include polyimide (PI), polyethylene terepthalate (PET), and polyethylene napthalate (PEN). PET and PEN are commercially available substrates produced by DuPont. Due to their semi-crystalline nature, PET and PEN possess lower coefficients of thermal expansion and may be heat-stabilized in order to expand processing temperature limits. This makes them attractive certain applications in which substrates will undergo exposure to elevated temperatures during device anode fabrication (e.g., sintering silver nanoparticles, PEDOT:PSS drying, etc.). FIG. 5 shows a comparison of properties between PEN and PET materials, taken from W. A. MacDonald, et al., Latest advances in substrates for flexible electronics, Journal of the SID, 15(12): 1075-1083, 2007. As shown, PEN has a higher temperature limit and is only marginally more rigid than PET.

As bio-centric electronic applications have gained traction in recent years, a new emphasis on skin-mountable electronics has led to many new materials and processing techniques. For certain embodiments, the ability to flex around a regular radius of curvature is important to maintain high-fidelity optical contact. A good substrate material would mimic the mechanical properties of human skin and adhere strongly to the skin while still permitting oxygen and moisture transport through the material. An epidermal electronics platform that bonds via van der Waals interactions between the human epidermis and the substrate material, polyester has been developed (D. H. Kim, et al. Epidermal Electronics. Science, 333(6044): 838-843, 2011.).

### Wirelessly-Linked Patch Network for Real-Time Monitoring Applications

In an embodiment, to control the LED, process information from the photodiode, and transmit this information to physicians; a variety of control and communication circuitry is provided.

### Circuit Technology Considerations

In certain embodiments, a board-level design including commercial off-the-shelf (COTS) components is used and/or an application-specific integrated circuit (ASIC) is used. There are several reasons that NIRS sensors are an attractive candidate for use of ASICs. One of the advantages of ASICs is their power efficiency. Unlike COTS solutions which are forced to use components with extra functionality (and thus extra power expenditure), ASICs may have leaner power budgets. One of the motivations to keep NIR sensors efficient is that low overall power usage and small transient current spikes make it possible to use printed batteries to power hematoma sensors. Printed batteries are an attractive power source because many people are hesitant to place conventional cell batteries on their skin due to the possibility of chemical leakage and heating. Moreover, since printed LEDs require 5 V and small cell batteries are typically 1.5 V, even with an efficient 1:2 on-chip DC-DC converter, it would require multiple batteries to power each sensor. Having multiple, rigid batteries detract from the sensors flexibility, which harms optical contact. Using layers of printed batteries on top of the printed LEDs/PDs advantageously allows the sensor to conform to the shape of the skull.

Not only does a low power ASIC allow for printed battery power, the reduced number of chips in comparison to a COTS board improves flexibility. For example, a typical wireless sensor might require requires ten or more chips as well as supporting passive components. Even if these chips were mounted on a flexible PCB, they would be much less flexible than the single chip needed by an ASIC solution as disclosed herein. Based on considerations of power efficiency, the ability to use printed batteries, and the reduced chip count; a fully ASIC NIR sensor interface will advantageously provide improved optical contact and will serve as an important innovation in the area of body sensor networks.

### Head-Mounted NIR Sensor Interface Circuitry

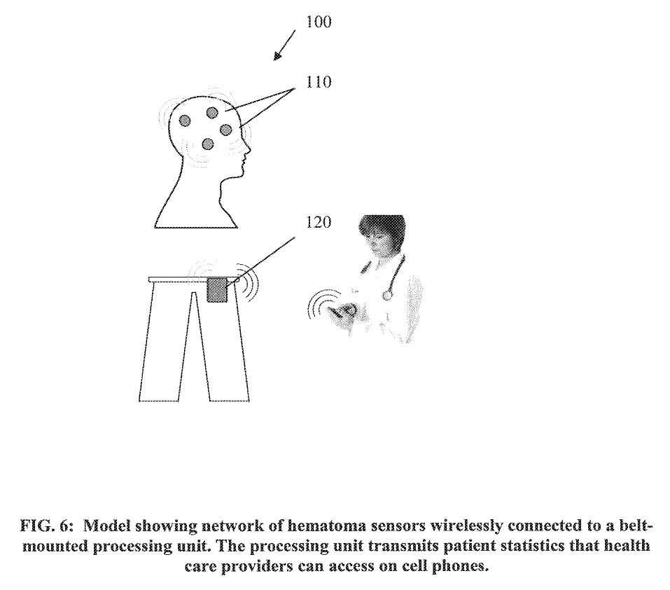

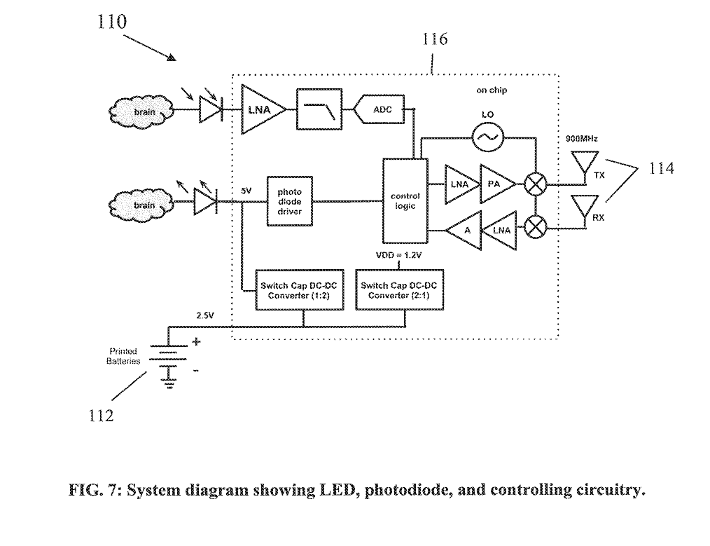

As shown in FIG. 6, an embodiment of a hematoma sensing network 100 includes two components: a network of small, head-mounted NIR sensors 110 and a processing unit 120 that receives information from the sensors 110. The processing unit 120 may be directly coupled to the sensors 110 or it may be remotely (e.g., wirelessly) coupled to the sensor 110. The processing unit 120 may transmit patient statistics that health care providers can access remotely, e.g., on cell phones or computer systems. As discussed above, the head-mounted sensor interface may be implemented in an ASIC including printed batteries, in one embodiment. FIG. 7 is a system-level diagram showing components of an NIR sensor 110, according to an embodiment. The following section will discuss the implementation of the power system, photodiode and LED interface circuitry 116, custom communication protocol, and antenna 114 that make up the sensor 110.

Power System:

Power is provided by a printed battery or batteries 112. In an embodiment, primary silver-zinc batteries are used. Such batteries have an energy density of about

$$4.1 \pm .3 \frac{mW}{{cm}^{2}}$$

at 1.5 V. To provide sufficient illumination, experiments indicate that printed LEDs operate at 5 V and dissipate 2-4 mW during operation. In addition to LEDs, the batteries should also power the sensor ASIC. The ASIC will dissipate on the order of 2.5 mW to 5 mW. Due to the step-down DC-DC converters, this is equivalent to a about 1.25 mW to 2.5 mW draw on the battery during transmission. One of the advantages hematoma sensors have over other body sensors (such as EEG) is they require a very low sampling frequency (on the order of 1 Hz). This means that power intensive processes such as LED pulsing and sensor transmission can happen very infrequently and at different times, which reduces the peak current drawn from batteries. In total, the maximum power that needs to be supplied by the batteries at any time is less than 5 mW and, as shown below, standby power is on the order of 10 μW, which is implementable using printed batteries.

To obtain a sufficiently high voltage to power the LEDs from 1.5 V batteries, in one embodiment, two batteries are used in series with an on-chip DC-DC voltage converter. Voltage doublers and halfers are efficient DC-DC converter configurations, achieving up to 80% efficiency. This configuration will provide 6 V, which is sufficient for powering LEDs. Moreover, a voltage halfer applied to a 3 V battery input provides a 1.5 V supply for the AISC.

Photodiode Interface

As shown in FIG. 7, components in the photo-diode interface for receiving and processing the light reflected from the brain include a low noise amplifier (LNA) that boosts the input photodiode signal. Because the sampling frequency may be low, the signal passes through a low pass filter to remove high frequency noise. An analog to digital converter (ADC) then digitizes the signal so it can be passed into a control unit for transmission.

Custom Communication Protocol

Once the signal has been digitized for transmission, it is necessary to use an appropriate communications protocol. For low transmission frequency/low power applications, a typical COTS solution is a ZigBee Rx/Tx chip. Texas Instruments provides an example of a ZigBee RF transceiver. One problem with this chip is it (and most ZigBee chips) require about 40 mW for receiving and 70 mW for transmitting, which is may not be feasible using printed batteries. Therefore, in one embodiment, a custom communication protocol operating at 900 MHz is used. A number of SOCs include custom communication protocols for body sensor networks at this frequency. For example, an OOK protocol in a 863-928 MHz band may be used.

In an embodiment, leveraging the fact that hematoma sensors need to transmit very infrequently, intelligent wakeup protocols are employed to reduce power consumption and extend battery life. For example, an always-on passive RF monitoring system to control sensor wakeup and low frequency transmission requests may be used. Using such a design, an active standby power consumption of less than 20 μW may be achieved. Using this sort of low power wakeup routine, it is possible to extend battery life with low power density printed batteries.

Antenna 114:

At 900 MHz, it is possible to make on-chip antennas. In one embodiment, however, a printed antenna is formed on the flexible substrate. In order to transmit a relatively isotropic signal 5-10 meters (which is more than sufficient for a body sensor network), an antenna footprint in the range of 2.8 cm to 4 cm can be used, which is well within the spatial boundaries of other printed components and can readily fit skull contours.

LED Controller:

In an embodiment, wakeup signals from the processing unit 120 are used to signal the beginning of a hematoma measurement. This measurement begins with LED activation. An LED control circuit as is well known may be implemented.

### Remote Data Processing Unit

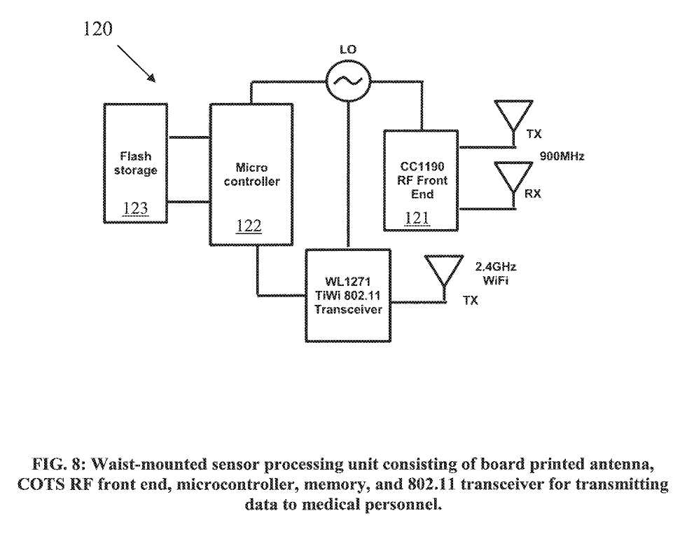

For a differential hematoma reading, multiple hematoma sensors 110 described above can be mounted on the head of a patient. Data from these sensors may be transmitted, e.g., at 900 MHz, to a larger, remote (e.g., waist-mounted) processing unit 120. A system level view of an embodiment of a processing unit 120 is shown in FIG. 8. PCB printed antennas receive signals from the sensors 110 and send the signals to an RF (e.g., COTS RF) front end 121 such as TIs Sub-1 GHz RF front end, which can receive in the 850-950 MHz band. A local oscillator (LO) provides a clock for the transceiver and a microcontroller 122, which performs signal processing such as identifying which sensors are transmitting, comparing data from these sensors to produce an OD reading, storing the data in a memory (e.g., flash memory) 123, and signaling the RF front end to transmit information to a cellular device, e.g., via a 802.11 protocol or other wireless protocol. This 802.11 transmission may be performed by a COTS wifi chip such as TIs WL1271 TiWi, which is designed for medical applications and has a transmit power of 20 dBm. This entire system can be powered by standard cell batteries.

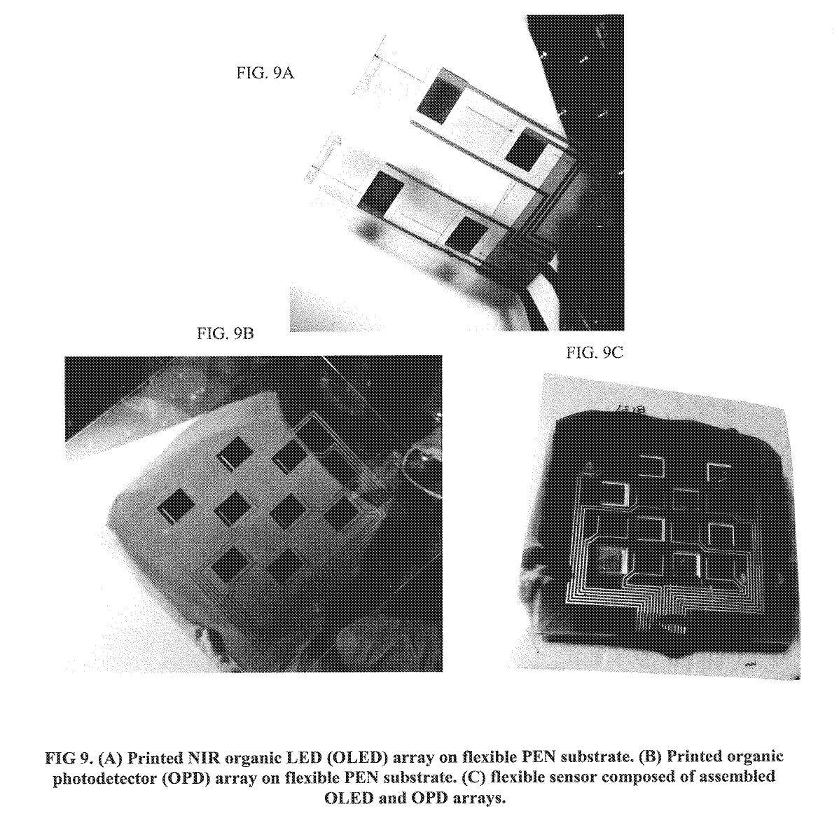

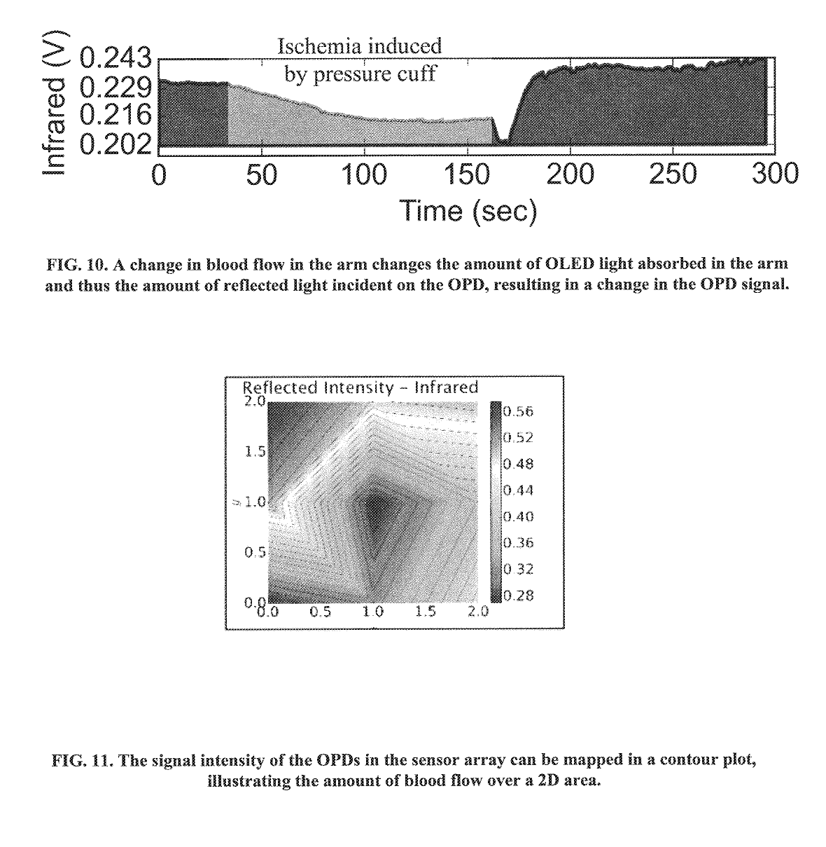

FIG. 9 shows an example of a fully assembled sensor composed of NIR OLEDs and OPDs that has been demonstrated to successfully sense a change in blood levels in a person's forearm when ischemia is induced by inflating a blood pressure cuff on the person's upper arm. FIG. 9A shows a printed NIR organic LED (OLED) array on flexible PEN substrate, FIG. 9B shows a printed organic photodetector (OPD) array on flexible PEN substrate, and FIG. 9C shows a flexible sensor composed of assembled OLED and OPD arrays. FIG. 10 shows an OPD signal upon excitation by light from NIR OLEDs that is reflected from the subject's arm. As the blood pressure cuff is inflated, the blood supply to the arm is cut-off, inducing ischemia; this is shown by the decrease in the OPD signal between approximately 40 and 160 s. At approximately 160 s, the blood pressure cuff is released and blood flow is restored to the arm, as shown by the increase in OPD signal. This demonstrates the ability of the flexible sensor composed of printed NIR OLEDs and OPDs to measure a change in blood flow in vivo, as is required for hematoma sensing. FIG. 11 shows how the reflected intensity of light at the sensor can vary over the area covered by each of its pixels. This functionality can be used to find the location of a hematoma on a person's skull.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Exemplary embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A hematoma detection device, comprising:
a flexible substrate;
an array of organic near infra-red (NIR) elements formed on the flexible substrate, wherein the array of NIR elements includes at least one organic NIR light emitting element and at least one organic NIR light detection element.

2. The device of claim 1, wherein the array of NIR elements includes at least one organic light emitting diode (LED) element and at least one organic photodetector (OPD) element.

3. The device of claim 2, wherein the NIR elements include an array of NIR LEDs and an array of NIR OPDs.

4. The device of claim 1, wherein the flexible substrate comprises a plastic material.

5. The device of claim 4, wherein the plastic material comprises one of a polyimide (PI) film, a polyethylene terephthalate (PET) film or a polyethylene naphthalate (PEN) film.

6. The device of claim 4, wherein the flexible substrate comprises a parylene layer formed on a flexible backing layer.

7. The device of claim 1, further including one or more printed batteries formed on the flexible substrate or on the array of organic NIR elements.

8. The device of claim 1, further including a transmitter and control element for wirelessly communicating with a remote device.

9. The device of claim 8, wherein the transmitter and control element includes a low noise amplifier, an analog to digital converter, and a wireless transmission circuit.

10. The device of claim 8, wherein the wireless transmission circuit includes or is coupled to a printed antenna element formed on the flexible substrate or on the array of organic NIR elements.

11. A hematoma sensor network system, comprising:
at least two wearable sensor devices, each wearable sensor device including:
an array of organic near infra-red (NIR) elements formed on a flexible substrate, wherein the array of NIR elements includes at least one organic NIR light emitting element and at least one organic NIR light detection element; and
a wireless communication element connected to the at least one organic NIR light detection element, wherein the communication element receives a signal from the at least one NIR light detection element and sends a corresponding sensor signal to a processing unit; and
the processing unit, wherein the processing unit receives the sensor signals from the at least two wearable sensor devices and stores the sensor signals and/or data derived from the sensor signals, to a memory.

12. The system of claim 11, wherein each array of NIR elements includes at least one organic light emitting diode (LED) element and at least one organic photodetector (OPD) element.

13. The system of claim 11, wherein each flexible substrate comprises a plastic material selected from the group consisting of a polyimide (PI) film, a polyethylene terephthalate (PET) film and a polyethylene naphthalate (PEN) film.

14. A method of forming a hematoma detection device, the method comprising:
providing a flexible substrate; and
forming an array of organic near infra-red (NIR) elements on the flexible substrate, wherein the array of NIR elements includes at least one organic NIR light emitting element and at least one organic NIR light detection element.

15. The method of claim 14, wherein forming an array includes printing said NIR elements on the flexible substrate.

16. The method of claim 15, wherein printing includes one or more of ink jet printing, screen printing and blade coating.

17. The method of claim 14, wherein the flexible substrate comprises a plastic material selected from the group consisting of a polyimide (PI) film, a polyethylene terephthalate (PET) film and a polyethylene naphthalate (PEN) film.

18. The method of claim 14, wherein forming an array includes using photolithography techniques to form said NIR elements on the flexible substrate.

19. A wearable sensor device, comprising:
a flexible substrate;
an array of organic near infra-red (NIR) elements formed on the flexible substrate, wherein the array of NIR elements includes at least one organic NIR light emitting element and at least one organic NIR light detection element.
